(19) United States
(12) Patent Application Publication
(10) Pub. No.: US 2008/0166262 A1
(43) Pub. Date: Jul. 10, 2008

(54) Medical packaging substrate with security feature

(76) Inventor: Ganesh C. Deka, Duluth, GA (US)

Correspondence Address: DORITY & MANNING, P.A., POST OFFICE BOX 1449, GREENVILLE, SC, 29602-1449, US

(21) Appl. No.: 11/649,714

(22) Filed: Jan. 4, 2007

(51) Int. Cl. A61L 2/20; B32B 3/24; C09J 5/00

(52) U.S. Cl. 422/28; 156/305; 428/131

(57) ABSTRACT

Fibrous webs having a visible security image and products constructed from such webs are generally disclosed. The fibrous webs having the security image also contain a binder composition that strengthens the web without significantly affecting the ability to see security image. The application of a binder composition to a watermarked or shadow marked web can be achieved without affecting the ability to see the security image formed by the watermark or shadow mark. In one embodiment, the present invention is generally directed to medical packaging substrates having security features, such as watermarks or shadow marks, in at least one surface of at least one fibrous web of the substrate.

### BACKGROUND OF THE INVENTION

In the past, watermarks or a shadow marks have been used to create security images in paper. Typically, watermarks and shadow marks are produced by inducing localized variations in the thickness of the cellulosic web. This variation in thickness, in turn, creates localized variations in the opacity of the paper, and so creates a contrast which makes the watermark visible, particularly in transmitted light. The desired localized variation in web thickness is typically effected by fiber displacement by means of a dandy roll which runs on top of the wet web on the wire of a Fourdrinier paper machine.

However, the variations in thickness of watermarked and shadow marked webs, though adding security features to the web, can adversely affect other properties of the web. For example, the porosity of the web can be affected by the variations in thickness. Likewise, the strength of the web can be affected by the variations in thickness of the web. These side-effects of the variations in thickness of watermarked and shadow marked webs result in these papers having limited uses in some applications where porosity and strength are important characteristics of the products constructed from the webs. As such, not all paper products have been able to utilize the advantages of security paper in order to help prevent counterfeiting of the paper product.

For example, one particular type of product that can be subject to counterfeiting, but has not to date utilized the advantages of security paper, is medical packaging substrates. Medical packaging substrates allow contents to be sterilized, protect contents during sterilization, and preserve their sterility upon subsequent storage until the packages are opened for use of the product. Such sterilization pouches and their usages are further described, for example, in U.S. Pat. No. 6,537,932, which is incorporated herein in its entirety by reference thereto for all purposes.

Due to the nature of the medical packaging substrate's use, counterfeit medical packaging substrates not only affect the sales of the copied products, but may also endanger medical technicians and their patients who are exposed to medical products sterilized and stored in cheap, counterfeit medical packaging substrates. Thus, it is important that the medical technicians can be confident that their medical products are sterilized and stored in quality medical packaging substrates.

As such, a need currently exists for an improved medical packaging substrate that includes a security feature that helps prevent counterfeiting and indicates the authenticity of the substrate.

### SUMMARY OF THE INVENTION

In one embodiment, the present invention is generally directed to a method for forming a nonwoven web. A suspension of fibers is formed into a fibrous web having a first thickness. The suspension contains a cellulosic fibrous material. A security image is formed in the fibrous web and is defined by portions of the web having a second thickness that differs from the first thickness such that the security image can be seen by a person. The fibrous web is impregnated with a binder composition. The binder composition can include a carboxylated polyacrylate. Also, in one embodiment, the binder composition can include an opacity modifier. In some embodiments, an organic dye can also be applied to at least one surface of the web.

The nonwoven web has a Gurley porosity of from about 0.1 to about 120 seconds per 100 cubic centimeters, such as from about 10 to about 80 seconds per 100 cubic centimeters or from about 20 to about 60 seconds per 100 cubic centimeters.

In one embodiment, the second thickness is greater than the first thickness such that the security image is a shadow mark that appears darker than the remainder of the web. In an alternative embodiment, the first thickness is greater than the second thickness such that the security image is a watermark that appears lighter than the remainder of the web.

In another embodiment, the present invention is generally directed to a medical packaging substrate including a fibrous web formed from a cellulosic fibrous material. The fibrous web has a first thickness and defines a security image having a second thickness that differs from the first thickness. The fibrous web is saturated with a latex saturant, resulting in a saturated fibrous web having a Gurley porosity of from about 0.1 to about 120 seconds per 100 cubic centimeters.

In yet another embodiment, the present invention is generally directed to a method for sterilizing an item. The item is sealed within a medical packaging substrate formed from a cellulosic fibrous material. The cellulosic fibrous material defines a fibrous web having a first thickness and also defines a security image having a second thickness that differs from the first thickness. The fibrous web is saturated with a latex saturant. The medical packaging substrate is contacted with a sterilizing gas.

Also, a method for forming a medical packaging substrate is generally disclosed. A suspension of fibers is formed into a fibrous web having a first thickness. The suspension contains a cellulosic fibrous material. A security image is formed in the fibrous web. The security image is defined by portions of the web having a second thickness that differs from the first thickness. The fibrous web is impregnated with a binder composition. The resulting nonwoven web has a Gurley porosity of from about 0.1 to about 120 seconds per 100 cubic centimeters.

Other features and aspects of the present invention are discussed in greater detail below.

### BRIEF DESCRIPTION OF THE DRAWINGS

FIG. 1 represents a cross-section of a watermarked fibrous web having a security image.

FIG. 2 represents a cross-section of a shadow marked fibrous web having a security image.

### DETAILED DESCRIPTION OF REPRESENTATIVE EMBODIMENTS

It is to be understood by one of ordinary skill in the art that the present discussion is a description of exemplary embodiments only, and is not intended as limiting the broader aspects of the present invention, which broader aspects are embodied in the exemplary construction.

Generally speaking, the present invention is directed to fibrous webs having a visible security image and products constructed from such webs. The fibrous webs having the security image also contain a binder composition that strengthens the web without significantly affecting the ability to see security image. Additionally, the present inventor has found that a fibrous web having watermark or shadow mark security images can be produced without sacrificing the strength and porosity qualities of the web.

In accordance with the present invention, a binder composition is applied to the fibers after web formation to improve the strength and surface properties of the web. For example, the binder composition can be applied to a formed wet web after the security image, such as a watermark or a shadow mark, is formed in the web by a dandy roll. The present inventor has surprisingly found that the application of a binder composition to a watermarked or shadow marked web can be achieved without affecting the ability to see the security image formed by the watermark or shadow mark.

As such, in one embodiment, the present invention is generally directed to medical packaging substrates having security features, such as watermarks or shadow marks, in at least one surface of at least one fibrous web of the substrate.

### Fibrous Webs Having a Security Image

The security images are generally defined by areas of the fibrous web that have a different thickness than the remainder of the web. For example, watermark images are defined by areas of the web that have a thinner thickness than the rest of the web. Thus, the watermark image appears as a lighter image in the web. On the other hand, shadow mark images are defined by areas of the web having a thicker thickness than the rest of the web. Thus, the shadow mark image appears as a darker image in the web.

Watermarks and shadow marks can be formed by the use of a dandy roll having either protrusions or recessions, respectively, in the outer surface of the roll. Thus, the dandy roll can displace the fibers of the web while the web is still in the wet state to create areas of differing thickness.

For example, watermarks can be formed by the use of a dandy roll having protrusions from the outer surface of the dandy roll. The protrusions, when contacting a wet web, displace fibers in the contact area, resulting in localized thinning of the paper. These localized thin areas form a security image in the web, which has reduced opacity and greater light transmittance than the remainder of the web. As such, the security image appears lighter than the remainder of the web to the naked eye when using transmitted light through the web. Exemplary processes of producing watermarks on a fibrous web are disclosed in U.S. Pat. No. 6,531,032 to Missell, et al., which is incorporated by reference.

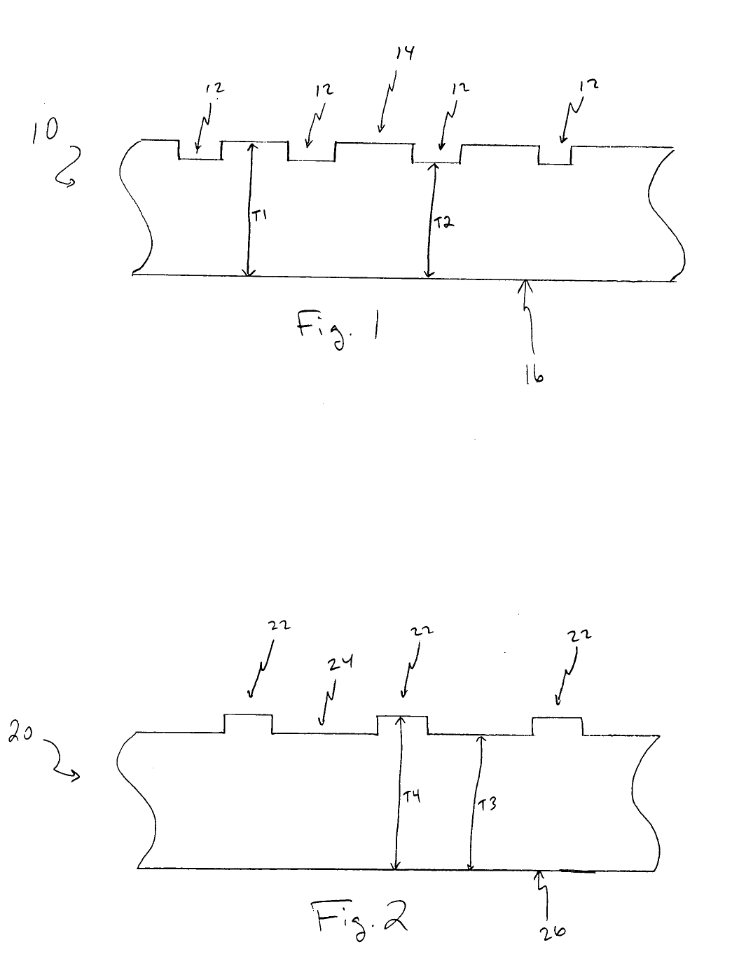

Referring to FIG. 1, for instance, a cross-section of a watermarked fibrous web 10 is shown having a security image formed by localized thin areas 12 in first outer surface 14. In this embodiment, the web 10 has a first thickness (T1) measured from the first outer surface 14 to the second outer surface 16. The localized thin areas 12 that form the watermark security image have a second thickness T2, measured from the outer surface of the thin area 12 to the second outer surface 16 of web 10, that is less than the first thickness T1. The variation in thickness between the first thickness T1 of the web 10 and the second thickness T2 of the localized thin areas 12 forming the watermark enable light to more easily transmit through the thin areas 12. For example, the thin areas 12 forming the watermark security image can transmit at least about 5% more light than the remainder of the web, such as at least about 10% more light. In one embodiment, the thin areas 12 forming the watermark security image can transmit from about 5% to about 35% more light than the remainder of the web, such as from about 10% to about 25% more light. Thus, the thin areas 12 form a security image that appears lighter than the remainder of the web 10 and can be seen by the naked eye, especially when utilizing transmitted light.

In contrast, shadow marks (sometimes referred to as “shaded marks,” “shaded watermarks,” or “shadow watermarks”) can be formed by the use of a dandy roll having recesses in the outer surface of the dandy roll. The recesses in the dandy roll, when contacting the wet web, results in fiber displacement that creates a localized thickening of the paper. These localized thick areas form a security image in the web, which has increased opacity and lesser light transmittance than the remainder of the web. As such, the security image appears darker than the remainder of the web to the naked eye when using transmitted light through the web.

Referring to FIG. 2, for instance, a cross-section of a shadow marked fibrous web 20 is shown having a security image formed by localized thick areas 22 in first outer surface 24. In this embodiment, the web 20 has a first thickness T3 measured from the first outer surface 24 to the second outer surface 26. The localized thick areas 22 that form the shadow mark security image have a second thickness T4, measured from the outer surface of the thick area 22 to the second outer surface 26 of web 20, that is greater than the first thickness T3. The variation in thickness between the first thickness T3 of the web 20 and the second thickness T4 of the localized thick areas 22 forming the shadow mark enable light to more easily transmit through the remainder of the web 20 when compared to the thick areas 22. For example, the remainder of the web 20 can transmit at least about 5% more light than the thick areas 22, such as at least about 10% more light. In one embodiment, the remainder of the web 20 can transmit from about 5% to about 35% more light than the thick areas 22, such as from about 10% to about 25% more light. Thus, the thick areas 22 form a security image that appears darker than the remainder of the web and can be seen in the web 20, especially when utilizing transmitted light.

No matter the method of formation the thickness variation in the fibrous web, the security image can be seen by the naked eye, especially when using transmitted light. The security image defined by the thickness variation can take on any form, design, character, shape, or other image visible on the surface of the web. For instance, the security image formed by the thickness variation in the web can be in the form of columns or rows that extend the length of the web. Also, the security image can repeat over the surface of the web.

The fibrous webs having a security image typically contain a cellulosic fibrous material. As used herein, the term “cellulosic fibrous material” generally refers to a material that contains wood based-pulps or other non-wood derived fiber sources. The pulp may be a primary fibrous material or a secondary fibrous material (“recycled”). Sources of pulp fibers include, by way of example, woods, such as softwoods and hardwoods; straws and grasses, such as rice, esparto, wheat, rye, and sabai; canes and reeds, such as bagasse; bamboos; woody stalks, such as jute, flax, kenaf, and cannabis; bast, such as linen and ramie; leaves, such as abaca and sisal; and seeds, such as cotton and cotton liners. Softwoods and hardwoods are the more commonly used sources of cellulose fibers. Examples of softwoods include, by way of illustration only longleaf pine, shortleaf pine, loblolly pine, slash pine, Southern pipe, black spruce, white spruce, jack pine, balsam fir, douglas fir, western hemlock, redwood, and red cedar. Examples of hardwoods include, again by way of illustration only, aspen, birch, beech, oak, maple, eucalyptus, and gum. Specific examples of such pulp fibers include softwood pulps available under the trade designation LL-19 from Neenah Paper, Inc. and INTERNATIONAL PINE® from International Paper Company. Other cellulosic fibers that may be used the present invention include eucalyptus fibers, such as Primacell Eucalyptus, available from Klabin Riocell, and other hardwood pulp fibers available under the trade designations LL-16 available from Neenah Paper, Inc., St. Croix hardwood available from Georgia-Pacific Corporation, and Leaf River hardwood available from Georgia-Pacific Corporation.

The pulp fibers may generally be chemical or mechanical pulp. Chemical pulp refers to fibrous materials from which most non-cellulose components are removed by chemical pulping without substantial mechanical post-treatment. Sulfite or sulfate (Kraft) chemical processes, for example, involve the dissolution of the lignin and hemi-cellulose components from the wood to varying degrees depending on the desired application. Mechanical pulp refers to fibrous materials made of wood processed by mechanical methods. Mechanical pulp is subdivided into the purely mechanical pulps (e.g., groundwood pulp and refiner mechanical pulp) and mechanical pulps subjected to chemical pretreatment (e.g., chemimechanical pulp or chemithermomechanical pulp). Synthetic cellulose-containing fibers may also be used, such as cellulosic esters, cellulosic ethers, cellulosic nitrates, cellulosic acetates, cellulosic acetate butyrates, ethyl cellulose, regenerated celluloses (e.g., viscose, rayon, etc.).

Although not required, the cellulosic fibrous material used to form the security paper of the present invention is typically a chemical pulp. Examples of such chemical pulps include, for instance, sulfite pulps, Kraft pulps (sulfate), soda pulps (cooked with sodium hydroxide), pulps from high-pressure cooking with organic solvents, and pulps from modified processes. Sulfite and Kraft pulps differ considerably in terms of their fibrous material properties. The individual fiber strengths of sulfite pulps are usually much lower than those of Kraft pulps. The mean pore width of the swollen fibers is also greater in sulfite pulps and the density of the cell wall is lower compared to Kraft pulps, which simultaneously means that the cell-wall volume is greater in sulfite pulps. Due to their higher strength, lower pore width, and higher density, Kraft pulps are typically employed in the present invention.

While the present invention has applicability to any of the above chemical pulping processes, it is particularly useful with the Kraft process and, as such, the Kraft process is described in more detail below. Initially, suitable trees are harvested, debarked and then chipped into suitable size flakes or chips. These wood chips are sorted with the small and the large chips being removed. The remaining suitable wood chips are then charged to a digester (vessel or tank for holding the chips and an aqueous digesting composition and which can be operated in either a batch or continuous mode). In a batch type digester, wood chips and a mixture of “weak black liquor”, the spent liquor from a previous digester cook, and “white liquor”, a solution of sodium hydroxide and sodium sulfide, which is either fresh or from the chemical recovery plant, is pumped into the digester. In the cooking process, lignin, which binds the wood fiber together, is dissolved in the white liquor forming pulp and black liquor. The digester is sealed and heated to a suitable cook temperature (e.g. up to about 180° C.) under high pressure. After an allotted cooking time at a particular temperature and pressure (H-factor) in the digester, its contents (pulp and black liquor) are transferred to a holding tank. The pulp in the holding tank is transferred to the brown stock washers while the liquid (black liquor formed in the digester) is sent to the black liquor recovery area. The black liquor is evaporated to a high solids content, usually 60-80% solids. Once cooked, the pulp is typically subjected to a bleaching process to delignify the material. Chlorine, chlorine dioxide, sodium hypochlorite, hydrogen peroxide, oxygen, and mixtures thereof, are employed in most conventional bleaching processes. Ozone is a particularly effective bleaching technique, and may be used to perform low consistency, medium consistency, or high consistency bleaching. Ozone bleaching is normally performed an acidic pH level (less than 7) to optimize delignification effectiveness.

Once cooked and optionally bleached, the raw cellulosic fibrous material is supplied for web formation in accordance with the present invention. Different cellulosic fibers may be selected to provide different attributes. The choice of fiber sources depends in part on the final application of the web. For example, softwood fibers may be included in the web to increase tensile strength. Hardwood fibers may be selected for their ability to improve formation or uniformity in distribution of the fibers. In one embodiment, the fibrous web may contain from about 30% to about 75% softwood fibers based on total dry weight of the fibers, and in some embodiments, from about 50% to about 75% softwood fibers based on total fiber dry weight. Likewise, the fibrous web may contain from about 25% to about 70% softwood fibers based on total dry weight of the fibers, and in some embodiments, from about 25% to about 50% softwood fibers based on total fiber dry weight.

If desired, synthetic fibers may also be used in conjunction with the cellulosic fibers to increase the tear resistance of the fibrous web. Examples of such synthetic fibers may include, for instance, polyolefins (e.g., polyethylene, polypropylene, polybutylene, etc.); polytetrafluoroethylene; polyesters (e.g., polyethylene terephthalate); polyvinyl acetate; polyvinyl chloride acetate; polyvinyl butyral; acrylic resins (e.g., polyacrylate, polymethylacrylate, polymethylmethacrylate, etc.); polyamides (e.g., nylon 6, nylon 6/6, nylon 4/6, nylon 11, nylon 12, nylon 6/10, and nylon 12/12); polyvinyl chloride; polyvinylidene chloride; polystyrene; polyvinyl alcohol; polyurethanes; polylactic acid; and so forth. The synthetic fibers may be monocomponent or multicomponent fibers. One example of a multicomponent fiber is comprised of two fibers having differing characteristics combined into a single fiber, commonly called a bicomponent or multicomponent fiber. Bicomponent fibers generally have a core and sheath structure where the core polymer has a higher melting point than the sheath polymer. Other bicomponent fiber structures and cross-sections, however, may be utilized. For example, bicomponent fibers may be formed with the two components residing in various side-by-side relationships as well as concentric and eccentric core and sheath configurations. One particular example of a suitable bicomponent fiber is available from KoSa under the designation CELBOND® T-255. CELBOND® T-255 is a synthetic polyester/polyethylene bicomponent fiber capable of adhering to cellulosic fibers when its outer sheath is melted at a temperature of approximately 128° C. When utilized, the synthetic fibers typically constitute from about 0.1% to about 30%, in some embodiments from about 0.1% to about 20%, and in some embodiments, from about 0.1% to about 10% of the dry weight of the web.

Particularly when natural fibers are employed, the fibrous material is generally placed in a conventional papermaking fiber stock prep beater or pulper containing a liquid, such as water. The fibrous material stock is typically kept in continued agitation such that it forms a suspension. If desired, the fibrous material may also be subjected to one or more refinement steps to provide a variety of benefits, including improvement of the bacterial filtration properties of the fibrous web. Refinement results in an increase in the surface area and amount of intimate contact of the fiber surfaces and may be performed using devices well known in the art, such as a disc refiner, a double disc refiner, a Jordan refiner, a Claflin refiner, or a Valley-type refiner. Various suitable refinement techniques are described, for example, in U.S. Pat. No. 5,573,640 to Frederick, et al., which is incorporated herein in its entirety by reference thereto for all purposes. The level of fiber degradation imparted by refinement may be characterized as “Canadian Standard Freeness” (CSF) (TAPPI Test Methods T-227 OM-94). For example, 800 CSF represents a relatively low amount of degradation, while 400 CSF represents a relatively high amount of degradation. In most embodiments of the present invention, the fibers are refined to about 400 to about 800 CSF, and in some embodiments, from about 600 CSF to about 750 CSF.

The resulting fibrous suspension may then be diluted and readied for formation into a fibrous web using conventional papermaking techniques. For example, the web may be formed by distributing the suspension onto a forming surface (e.g., wire) and then removing water from the distributed suspension to form the web. This process may involve transferring the suspension to a dump chest, machine chest, clean stock chest, low density cleaner, headbox, etc., as is well known in the art. Upon formation, the fibrous web may then be dried using any known technique, such as by using convection ovens, radiant heat, infrared radiation, forced air ovens, and heated rolls or cans. Drying may also be performed by air drying without the addition of thermal energy. If desired, the fibers may be treated with the pH modifier at any stage of the papermaking process.

Additionally, other additives may also be applied to the fibers. For example, wet-strength agents may be used to improve the strength properties of the web during formation. The wet-strength agent may be present in an amount from about 0.001 wt. % to about 5 wt. %, in some embodiments from about 0.01 wt. % to about 2 wt. %, and in some embodiments, from about 0.1 wt. % to about 1 wt. %, based on the dry weight of the fibers. Wet strength agents are typically water soluble, cationic oligomeric or polymeric resins that are capable of bonding with the cellulosic fibers. Suitable polyamine-epichlorohydrin, polyamide epichlorohydrin or polyamide-amine epichlorohydrin resins (PAE resins) are available from Hercules, Inc. of Wilmington, Del. under the designation “KYMENE®” (e.g., KYMENE® 557H or 557 LX or 613). KYMENE® 557 LX and KYMENE® 613, for example, are polyamide epicholorohydrin polymers that contain both cationic sites, which may form ionic bonds with anionic groups on the pulp fibers, and azetidinium groups, which may form covalent bonds with carboxyl groups on the pulp fibers and crosslink with the polymer backbone when cured. Other suitable polyamide-epichlorohydrin resins are described in U.S. Pat. No. 3,885,158 to Petrovich; U.S. Pat. No. 3,899,388 to Petrovich; U.S. Pat. No. 4,129,528 to Petrovich; U.S. Pat. No. 4,147,586 to Petrovich; and U.S. Pat. No. 4,222,921 to van Eanam, which are incorporated herein in their entirety by reference thereto for all purposes.

Of course, other wet strength agents may also be employed in certain embodiments of the present invention. For example, other suitable wet strength agents may include dialdehyde starch, polyethylene imine, mannogalactan gum, glyoxal, and dialdehyde mannogalactan. Particularly useful wet-strength agents are water-soluble polyacrylamide resins available from Cytec Industries, Inc. of West Patterson, N.J. under the designation PAREZ® (e.g., PAREZ® 631 NC). The PAREZ® resins are formed from a polyacrylamide-glyoxal polymer that contains cationic hemiacetal sites. These sites may form ionic bonds with carboxyl or hydroxyl groups present on the cellulosic fibers to provide increased strength to the web. Because the hemiacetal groups are readily hydrolyzed, the wet strength provided by the resins is primarily temporary. Such resins are believed to be described in U.S. Pat. No. 3,556,932 to Coscia, et al. and U.S. Pat. No. 3,556,933 to Williams, et al., which are incorporated herein in their entirety by reference thereto for all purposes.

If desired, the fibrous web may also have applied an adhesive coating to enhance peel strength, decrease permeability, and/or increase the bacteria barrier. The coating may include any known adhesive, including pressure-sensitive or hot-melt adhesives.

In addition to the ingredients set forth above, various other additives may also be employed in the fibrous web. The additives may be applied directly to the web or fibers, in conjunction with the binder composition or adhesive coating, or as a separate coating. By way of example, suitable additives may include antifoaming agents, pigments, processing aids, and dispersing agents. Examples of antifoaming agents include, but are not limited to, products such as NALCO® 7518 available from Nalco Chemical Company or DOW Corning® Antifoam available from Dow Corning Corporation. Dispersing agents or surfactants include, but are not limited to, products such as TAMOL® 731A available from Rohm & Haas Co., PLURONIC® F108 available from BASF Corporation, SMA® 1440 Resin available from ATOFINA Chemicals, Inc., and TERGITOL® 15S available from Union Carbide Corp. Examples of processing aids may include, but are not limited to, products such as NOPCOTE® DC-100A available from Geo Specialty Chemicals, Inc., SCRIPSET® 540 available from Solutia, Inc. and AQUAPEL® 752 available from Hercules Incorporated. Examples of pigments used to increase opacity include but are not limited to, titanium dioxide such as TI-PURE® Rutile Titanium Dioxide available from E.I. Du Pont De Nemours & Co. and kaolin pigments, which are available from a variety of manufacturers. A wide range of pigments and dyes may also be added to impart color to the saturated sheet. The foregoing list of categories of additives and examples of categories is provided by way of example and is not intended to be exhaustive.

The porosity of the web, after the security image is formed on the web, can be sufficient to allow for good pick up of the binder composition, when it is later applied. For example, the porosity of the web, prior to application of a binder composition but after forming a security image, can be less than about 30 seconds per 100 cubic centimeters, such as from about 0.1 to about 25 seconds per 100 cubic centimeters or from about 10 to about 25 seconds per 100 cubic centimeters, as measured by the Gurley porosity method, TAPPI standard procedure number TAPPI Test Method No. T 460 om-96 (1996), as explained in greater detail below.

### Binder Composition

As explained above, a binder composition is applied to the fibrous web, either before or after the security image is imparted on the web. For example, in one embodiment, the web can first be wet laid, then the security image formed on the wet web, then the web can be dried, and finally a binder composition can be applied to at least one surface of the dried web having a security image.

Typically, the binder composition includes a latex polymers, such as polyacrylates, including polymethacrylates, poly(acrylic acid), poly(methacrylic acid), and copolymers of the various acrylate and methacrylate esters and the free acids; styrene-butadiene copolymers; ethylene-vinyl acetate copolymers; nitrile rubbers or acrylonitrile-butadiene copolymers; poly(vinyl chloride); poly(vinyl acetate); ethylene-acrylate copolymers; vinyl acetate-acrylate copolymers; neoprene rubbers or trans-1,4-polychloroprenes; cis-1,4-polyisoprenes; butadiene rubbers or cis- and trans-1,4-polybutadienes; and ethylene-propylene copolymers. For example, many conventional latex polymers are reacted with N-methylol acrylamide, N-(n-butoxy methyl) acrylamide, N-(iso-butoxy methyl) acrylamide, N-methylol methacrylamide, and other similar crosslinking agents. Also, latex polymers containing carboxyl functional groups can be utilized, such as carboxylated (carboxy-containing) polyacrylates, carboxylated nitrile-butadiene copolymers, carboxylated styrene-butadiene copolymers, carboxylated ethylene-vinylacetate copolymers, and polyurethanes. Specific examples of suitable carboxylated, formaldehyde-free latex polymers are polyacrylate binders available under the designations HYCAR® 26469, 26552, and 26703 from Noveon, Inc. of Cleveland, Ohio. The carboxylated latex polymer may be self-crosslinking. Alternatively, a crosslinking agent may be employed that is reactive to the carboxyl groups without releasing formaldehyde. One example of such a crosslinking agent is an aziridine oligomer having at least two aziridine functional groups, such as XAMA®-7 (Noveon, Inc. of Cleveland, Ohio) and Chemitite PZ-33 (Nippon Shokubai Co. of Osaka, Japan).

In addition to a latex polymer, the binder composition may also contain a heat-sealable polymer to help improve the peel strength of the resulting medical package during use. Examples of such heat-sealable polymers include, but are not limited to, homopolymers and heteropolymers of lower alkenes, e.g., ethylene and/or propylene. Specific examples of such heat-sealable polymers are polyethylene, polypropylene, ethylene acrylic acid, and ethylene vinyl acetate. One particularly desirable heat-sealable polymer is ethylene acrylic acid, such as commercially available under the name “Michem® Prime 4983R” from Michelman, Inc. Michem® Prime 4983R is a dispersion of Dow PRIMACOR® 59801 (copolymer of ethylene and acrylic acid that has an ethylene content of approximately 80%). Other suitable heat-sealable polymers may be described in U.S. Pat. No. 6,887,537 to Bean, et al., which is incorporated herein in its entirety by reference thereto for all purposes. When employed, heat-sealable polymers may constitute from about 35 wt. % to about 85 wt. % based on the total weight of the solids of the binder composition, in some embodiments, from about 40 wt. % to about 70 wt. %, and in some embodiments, from about 50 wt. % to about 60 wt. % of the binder composition. Likewise, latex polymers may constitute from about 25 wt. % to about 75 wt. %, in some embodiments from about 30 wt. % to about 60 wt. %, and in some embodiments, from about 40 wt. % to about 50 wt. % of the binder composition.

The binder composition may be applied to the cellulosic fibrous material before, during, and/or after web formation using any technique known in the art. Preferably, the binder composition is impregnated into the fibrous web using suitable techniques for impregnating a web with a binder composition described in U.S. Pat. No. 5,595,828 to Weber and U.S. Patent Application Publication No. 2002/0168508 to Reed, et al., which are incorporated herein in their entirety by reference thereto for all purposes. The amount of the binder composition applied may vary depending on the desired properties of the web, such as the desired permeability. Typically, the binder composition is present at an add-on level of from about 10% to about 90%, in some embodiments from about 20% to about 70%, and in some embodiments, from about 30% to about 60%, such as about 30% to about 40%. The add-on level is calculated, on a dry weight basis, by dividing the dry weight of binder composition applied by the dry weight of the web before treatment, and multiplying the result by 100.

In one particular embodiment, the binder composition can include an opacity modifier to control the prominence of the security image. For example, adding more of the opacity modify to the binder composition can reduce the ability to see the security image in the fibrous web, thus reducing the prominence of the security image in the web. As such, the security image may be somewhat hidden in the web, but still capable of being seen by the naked eye, especially with the use of transmitted light. For example, when the web having a security image is held between a light source and the eye, the security image remains distinctly visible, even if it is somewhat hidden in the web.

For instance, the opacity modifier can be any material that can decrease the overall opacity of the web. Suitable opacity modifiers can include, without limitation, pigments, clays, or precipitated opaque salts (such as calcinated clay and calcium carbonate). For example, the pigment may include titanium dioxide (TiO₂) or other solids capable of light reflecting or light absorbing.

The opacity modifier can be present in the binder composition in any amount, as long as the security image in the resulting impregnated web is still visible, even if only through the use of transmitted light. For example, the opacity modifier can be present in the binder composition up to about 35 parts per 100 dry parts of the latex, such as from about 5 to about 25 parts per 100 dry parts of the latex or from about 10 to about 20 parts per 100 dry parts of the latex.

### Organic Dyes

In addition to the binder composition, an organic dye can be applied, in some embodiments, to the fibrous web having a security image. In one embodiment, the organic dye can be incorporated within the binder composition. Alternatively, the organic dye can be applied to the web either before or after application of the binder composition. For instance, the dye can be included in the fibrous slurry in used to form the web.

According to this embodiment, application of the organic dye to the fibrous web does not adversely affect the ability to see the security image in the web. For example, a fibrous web containing a shadow mark security image can be dyed with a binder composition containing an organic dye. However, the darker shadow mark security image can still be seen in the web.

For example, the organic dye can include, by way of illustration only, triarylmethyl dyes, such as Malachite Green Carbinol base {4-(dimethylamino)-α-[4-(dimethylamino)phenyl]-α-phenylbenzene-methanol}, Malachite Green Carbinol hydrochloride {N-4-[[4-(dimethylamino)phenyl]phenylmethylene]-2,5-cyclohexyldien-1-ylide ne]-N-methyl-methanaminium chloride or bis[p-(dimethylamino)phenyl]phenylmethylium chloride}, and Malachite Green oxalate {N-4-[[4-(dimethylamino)phenyl]phenylmethylene]-2,5-cyclohexyldien-1-ylide ne]-N-methylmethanaminium chloride or bis [p-(dimethylamino)phenyl]phenylmethylium oxalate}; monoazo dyes, such as Cyanine Black, Chrysoidine [Basic Orange 2; 4-(phenylazo)-1,3-benzenediamine monohydrochloride], Victoria Pure Blue BO, Victoria Pure Blue B, basic fuschin and β-Naphthol Orange; thiazine dyes, such as Methylene Green, zinc chloride double salt [3,7-bis(dimethylamino)-6-nitrophenothiazin-5-ium chloride, zinc chloride double salt]; oxazine dyes, such as Lumichrome (7,8-dimethylalloxazine); naphthalimide dyes, such as Lucifer Yellow CH {6-amino-2-[(hydrazinocarbonyl)amino]-2,3-dihydro-1,3-dioxo-1H-benz[de]iso quinoline-5,8-disulfonic acid dilithium salt}; azine dyes, such as Janus Green B {3-(diethylamino)-7-[[4-(dimethylamino)phenyl]azo]-5-phenylphenazinium chloride}; cyanine dyes, such as Indocyanine Green {Cardio-Green or Fox Green; 2-[7-[1,3-dihydro-1,1-dimethyl-3-(4-sulfobutyl)-2H-benz [e]indol-2-ylidene]-1,3,5-heptatrienyl]-1,1-dimethyl-3-(4-sulfobutyl)-1H-b enz[e]indolium hydroxide inner salt sodium salt}; indigo dyes, such as Indigo {Indigo Blue or Vat Blue 1; 2-(1,3-dihydro-3-oxo-2H-indol-2-ylidene)-1,2-dihydro-3H-indol-3-one}; coumarin dyes, such as 7-hydroxy-4-methylcoumarin (4-methylumbelliferone); benzimidazole dyes, such as Hoechst 33258 [bisbenzimide or 2-(4-hydroxyphenyl)-5-(4-methyl-1-piperazinyl)-2,5-bi-1H-benzimidazole trihydrochloride pentahydrate]; paraquinoidal dyes, such as Hematoxylin {Natural Black 1; 7,11b-dihydrobenz[b]indeno[1,2-d]pyran-3,4,6a,9,10(6H)-pentol}; fluorescein dyes, such as Fluoresceinamine (5-aminofluorescein); diazonium salt dyes, such as Diazo Red RC (Azoic Diazo No. 10 or Fast Red RC salt; 2-methoxy-5-chlorobenzenediazonium chloride, zinc chloride double salt); azoic diazo dyes, such as Fast Blue BB salt (Azoic Diazo No. 20; 4-benzoylamino-2,5-diethoxybenzene diazonium chloride, zinc chloride double salt); phenylenediamine dyes, such as Disperse Yellow 9 [N-(2,4-dinitrophenyl)-1,4-phenylenediamine or Solvent Orange 53]; diazo dyes, such as Disperse Orange 13 [Solvent Orange 52; 1-phenylazo-4-(4-hydroxyphenylazo)naphthalene]; anthraquinone dyes, such as Disperse Blue 3 [Celliton Fast Blue FFR; 1-methylamino-4-(2-hydroxyethylamino)-9,10-anthraquinone], Disperse Blue 14 [Celliton Fast Blue B; 1,4-bis(methylamino)-9,10-anthraquinone], and Alizarin Blue Black B (Mordant Black 13); trisazo dyes, such as Direct Blue 71 {Benzo Light Blue FFL or Sirius Light Blue BRR; 3-[(4-[(4-[(6-amino-1-hydroxy-3-sulfo-2-naphthalenyl)azo]-6-sulfo-1-naphth alenyl)azo]-1-naphthalenyl)azo]-1,5-naphthalenyl)azo]1,5-naphthalenedisulfo nic acid tetrasodium salt}; xanthene dyes, such as 2,7-dichlorofluorescein; proflavine dyes, such as 3,6-diaminoacridine hemisulfate (Proflavine); sulfonaphthalein dyes, such as Cresol Red (o-cresolsulfonaphthalein); phthalocyanine dyes, such as Copper Phthalocyanine {Pigment Blue 15; (SP-4-1)-[29H, 31H-phthalocyanato(2-)-N₂₉,N₃₀,N₃₁,N₃₂ ]copper}; carotenoid dyes, such as trans-β-carotene (Food Orange 5); carminic acid dyes, such as Carmine, the aluminum or calcium-aluminum lake of carminic acid (7-a-D-glucopyranosyl-9,10-dihydro-3,5,6,8-tetrahydroxy-1-methyl-9,10-dioxo-2-anthracenecarbonylic acid); azure dyes, such as Azure A [3-amino-7-(dimethylamino)phenothiazin-5-ium chloride or 7-(dimethylamino)-3-imino-3H-phenothiazine hydrochloride]; and acridine dyes, such as Acridine Orange [Basic Orange 14; 3,8-bis(dimethylamino)acridine hydrochloride, zinc chloride double salt] and Acriflavine (Acriflavine neutral; 3,6-diamino-10-methylacridinium chloride mixture with 3,6-acridinediamine).

The organic dye, when applied separately from the binder composition, can be applied according to any method. For example, the organic dye can be substantially uniformly applied to the paper web having a security image. Alternatively, the organic dye can be applied in the form of a design or other image to the web.

### Medical Packaging Substrate

The fibrous web of the present invention may be utilized as a sterilization package in any manner known to those skilled in the art. For example, a web having a security image may be sealed to a base component using a heat seal device that applies heat to the edges or surfaces of the web and base component (optionally, in conjunction with an adhesive) to form a pouch, rigid container (e.g., tub or tray), etc. Typical materials used for the base component include, but are not limited to, nylon, polyester, polypropylene, polyethylene (e.g., low density, linear low density, ultra low density and high density polyethylene), and polystyrene. Examples of such packages are described, for instance, in U.S. Pat. No. 3,991,881 to Augurt; U.S. Pat. No. 4,183,431 to Schmidt, et al.; U.S. Pat. No. 5,217,772 to Brown, et al.; and U.S. Pat. No. 5,418,022 to Anderson, et al., which are incorporated herein in their entirety by reference thereto for all purposes.

Regardless of the particular manner in which it is formed, the fibrous web having security features, such as watermarks or shadow marks, possesses certain characteristics that facilitate its use in sterilization processes. For example, the permeability of the web (with optional coatings) is generally high enough to allow for the flow of gases during sterilization, but not so high as to significantly increase the ability of bacteria or other pathogens to penetrate through the web. One indicator of the permeability of a web is “Gurley porosity”, which is determined in accordance with TAPPI Test Method No. T 460 om-96 (1996). High Gurley porosity values correspond to low web permeability, and low Gurley porosity values likewise correspond to high web permeability. When used as a medical packaging substrate, the web of the present invention typically has a Gurley porosity of up to about 120 seconds per 100 cubic centimeters, in some embodiments from about 10 to about 80 seconds per 100 cubic centimeters, and in some embodiments, from about 20 to about 60 seconds per 100 cubic centimeters. As readily understood by those skilled in the art, the porosity of the web may be achieved through modification of a variety of parameters, including the type and amount of the binder composition, the type and weight of the fibrous web, and so forth. Also, it is understood that these porosity values are of the web as a whole. As pointed out above, the security features of the web result in varying thickness in the web, which can create a web having localized variances in porosity throughout the surface area of the web.

Further, the medical packaging substrate of the present invention also exhibits good barrier efficacy to bacteria, as expressed by percent bacterial filtration efficiency (“BFE”). The percent BFE generally represents the ability of a sample to act as a barrier to microorganisms and has an upper limit of 100%, which indicates that 100% of the microorganisms were intercepted by the test material. Typically, the percent BFE of the medical packaging substrate of the present invention is at least about 95%, in some embodiments at least about 97%, and in some embodiments, at least about 99%. Another parameter that is indicative of the barrier efficacy of the medical packaging substrate of the present invention is the log reduction value (“LRV”). LRV is the difference, measured in log scale, between the number of colony forming units (“CFU”) on a control media and the number of CFU on a test media. The range of measurable LRV is generally between 0 to 5, where higher numbers indicate greater barrier efficacy. The number of colony forming units may be measured in accordance with ASTM F 1608-95. Typically, the medical packaging substrate of the present invention exhibits a LRV of at least about 3, in some embodiments at least about 4, and in some embodiments, about 5. A more detailed description of the manner in which % BFE and LRV are determined is provided in U.S. Pat. No. 6,887,537 to Bean, et al.

The contents of the packaging may generally vary as is well known in the art and may include, for instance, surgical devices, implants, tubing, valves, gauzing, syringes, protective clothing (e.g., surgical gowns, drapes and gloves), or any other sterilizable item. Once the packaging is provided with the desired contents, it is then subjected to gas sterilization. Any suitable gas can be used during the gas sterilization. In one particular embodiment, for instance, the gas can be ethylene oxide. Various gas sterilization techniques may be utilized in the present invention. For examples, several suitable gas sterilization techniques are described in U.S. Pat. No. 5,069,880 to Karlson; U.S. Pat. No. 5,868,999 to Karlson; and U.S. Pat. No. 6,365,103 to Fournier, as well as U.S. Patent Application Publication No. 2002/0085950 to Robitaille, et al., all of which are incorporated herein in their entirety by reference thereto for all purposes.

The present invention may be better understood with reference to the following examples.

### EXAMPLES

All the following samples and examples thereof are based on paper produced in a Fourdrinier paper machine. Both softwood pulp (from Hinton) and Maple hardwood pulp (Sappi) were used. Whereas samples 1 is based on 100% softwood Hinton pulp, sample 2 is based on pulp with both hardwood and softwood. A shadow mark using a design available on paper cup insulters for coffee from Starbuck was produced on the paper using a dandy roll. The samples were compared to a control paper sheet having no shadow marking.

Shadow marking can be effectively produced by controlling variables such as (i) increased refining of pulp (ii) decreasing softwood content in a hardwood+softwood formulation (iii) with stock at lower consistency as it approaches the dandy roll etc. However, increased refining reduces porosity of the paper and thereby reduces porosity of the paper. And this in turn will make the web tighter preventing sterilization. Increasing hardwood will also reduce porosity and thereby retard sterilization. Therefore, to form a substrate that is suitable for medical packaging, it requires combination of very low intensity refining, optimum hardwood and softwood pulp combinations in the furnish as well as adequate water in the pulp as it approaches dandy roll for shadow mark.

It may also be noted that clarity of the shadow mark is also dependent on the subsequent binder formulation and constituents thereof. For example, one particular ingredient that effected clarity of the shadow mark is the amount of TiO₂ used in this case for opacity control. An optimum level of TiO₂ is added to the formulation for clarity of the shadow marks.

Table 1 shows the properties of shadow marked paper made with 100% softwood (sample 1) and [50% softwood+50% hardwood] (sample 2) as well as control sample without any shadow mark:

TABLE 1

| | Sample 1 | Sample 2 | Control |
|---|---|---|---|
| Pulp | 100% softwood | 50% softwood, | 100% softwood |
| | | 50% hardwood | |
| Refiner Setting | 36 amp - Jordan | 0 amp - Jordan | |
| | 40 amp - Disc | 40 amp - Disc | |
| Shadow Marking | Good | Good | Not applicable |
| Dry Basis Weight | 26.2 | 20.5 | 20.2 |
| (lbs/1300 sq.ft.) | | | |
| Caliper (mils) | 5.7 | 5.8 | 6.9 |
| Gurley Porosity, | 36.4 | 19.3 | 9.0 |
| 8 sheets | | | |
| (sec/100 sec) | | | |
| Gurley Porosity, | 5.3 | 2.6 | 1.2 |
| 1 sheet | | | |
| (sec/100 sec) | | | |
| Dry Tensile | 9.5 | 7.0 | 5.6 |
| Strength, MD | | | |
| (kg/15 mm) | | | |
| Dry Tensile | 4.4 | 3.7 | 3.3 |
| Strength, CD | | | |
| (kg/15 mm) | | | |
| Dry Stretch (%), | 1.56 | 1.19 | 1.37 |
| MD | | | |
| Dry Stretch (%), | 4.68 | 3.94 | 3.25 |
| CD | | | |
| Maximum Pore | 32.0 | 29.0 | 51.8 |
| Size, μm | | | |

It may be noted that even with 100% softwood and with light refining, the porosity number in sample 1 is much higher than sample 2. Thus, saturation of sample 1 was very difficult.

Each of the examples 1-5 below were prepared from paper samples 1 and 2 above. Examples 1 through 4 were saturated using laboratory saturator, while example 5 was saturated using Faustell Horizontal Type treater from Morrison Textile Machinery Company having overall length of 2 feet 7 inches and inside roll diameter of 1 foot 11 inches. A continuous web 10″ wide was saturated in this treater with binder formulation.

### Example 1

The paper made with 50% softwood pulp and 50% hardwood pulp (Sample 2) was saturated with the saturant of Table 2.

TABLE 2

| | % Solids of | | |
|---|---|---|---|
| Ingredient | ingredient | Dry Parts | Wet parts |
| Water | NA | NA | 49 |
| Hycar 26469 | 48 | 50 | 104.2 |
| TiO₂ | 55 | 10 | 18.2 |
| total | 35.6 | 60 | 171.4 |

It had a wet pickup of 6.3 grams, a dry pickup of 2.24 grams, and an add-on of 42.4%. The saturated paper of Sample 2 had the following properties, shown in Table 3:

TABLE 3

| | Saturated |
|---|---|
| | Sample 2 |
| Dry Basis Weight (lbs/1300 sq.ft.) | 29.4 |
| Caliper (mils) | 5.4 |
| Gurley Porosity, | 132.7 |
| 8 sheets (sec/100 sec) | |
| Gurley Porosity, | 18.9 |
| 1 sheet (sec/100 sec) | |
| Maximum Pore Size (μm) | 29 |
| Dry Tensile Strength, MD | 13.3 |
| (kg/15 mm) | |
| Dry Tensile Strength, CD | 8.3 |
| (kg/15 mm) | |
| Dry Stretch (%), MD | 2.82 |
| Dry Stretch (%), CD | 7.00 |
| Wet Tensile Strength, MD | 2.2 |
| (kg/15 mm) | |
| Wet Tensile Strength, CD | 1.3 |
| (kg/15 mm) | |
| Wet Stretch (%), MD | 2.69 |
| Wet Stretch (%), CD | 6.31 |

The paper made with 100% softwood pulp (sample 1) was difficult to saturate and could not be effectively saturated.

### Example 2

The paper made with 50% softwood pulp and 50% hardwood pulp (Sample 2) was saturated with the saturant of Table 4.

TABLE 4

| | % Solids of | | |
|---|---|---|---|
| Ingredient | ingredient | Parts | Wet parts |
| water | | | 3100 |
| Rhoplex B-20 | 46 | 100 | 1739 |
| Nalco 7518, | | | 0.9, 0.9 |
| water | | | |
| Ammonia, | 19 | 0.5 | 21.1, 14.3 |
| water | | | |
| Aquapel 752 | 15 | 2 | 107 |
| Black LFS s.s. | 25 | 12 | 384 |
| Total | 17.0 | | 5367 |

The saturated paper had a wet pickup of 6.57 grams, a dry pickup of 1.12 grams, and an add-on of 19.75%.

The Black LFS stock solution (s.s.) was prepared from Black LFS Powder Dye added to water to form a solution having 25% solids.

### Example 3

Sample 2 was saturated with a saturant having the following composition shown in Table 5:

TABLE 5

| | % Solids of | | |
|---|---|---|---|
| Ingredient | ingredient | Parts | Wet parts |
| Water | | | 52.85 |
| Hycar 1562 × 28 | 40.57 | 60.85 | 150 |
| Total | 29.83 | 60.85 | 202.85 |

The paper made had a wet pickup of 5.15 grams, a dry pickup of 1.46 grams, and an add-on of 29.6%.

The shadow mark was still visible.

### Example 4

Sample 2 was saturated with a saturant having the following composition shown in Table 6:

TABLE 6

| | % Solids of | | |
|---|---|---|---|
| Ingredient | ingredient | Parts | Wet parts |
| water | | | 138.1 |
| Hycar | 40.57 | 81.14 | 200 |
| 1562 × 28 | | | |
| Total | 24.36 | 81.14 | 338.1 |

The paper made with 50% softwood pulp and 50% hardwood pulp (Sample 2) was saturated with the saturant of Table 6 and had a wet pickup of 5.78 grams, a dry pickup of 1.41 grams, and an add-on of 26.1%.

The shadow mark was still visible.

### Example 5

As mentioned earlier, example 5 is based on saturating sample 2 Fourdrinier paper on a Faustel treater. At the beginning of the run, basis weight and porosity of the paper (in roll form) were measured as follows:

Basis weight: 20.4 lbs/1300 square feet.

Gurley Porosity, 1 sheet: 3.9 sec/100 cc
8 sheets: 36.5 sec/100 cc

It was saturated by a Faustel using the following saturant formulation, as shown in Table 7:

TABLE 7

| Ingredient | % | Dry Parts | Wet Parts |
|---|---|---|---|
| Water | | | 51.1 |
| Hycar 26469 | 48.5 | 100 | 206.2 |
| Ammonia | 28.0 | 0.4 | 1.43 |
| PD-14 | 52.1 | 20 | 38.4 |
| Total | 40.5 | 120.4 | 297.1 |

The saturant was diluted to 33.9% solid and ran in the Faustel. At the beginning of the run, the pick-up % of the saturant was 31.2, and at the end of the run, the pick-up % of the saturant was 32.8%.

The saturated paper was calendered using laboratory steel calender with steel rolls and ran under 10 psi loading and with single pass. Sheet samples were sent to Nelson Laboratories, 6280 Southern Redwood Road, Salt Lake City, Utah 84123-6600 for BFE & LRV testing. Sheet samples were also sent to Ethox, 251 Seneca Street, Buffalo, N.Y. 14204 for LRV testing.

The results are shown in Table 8 as follows:

TABLE 8

| | | | | | Spore | LRV |
|---|---|---|---|---|---|---|
| | Filter | | Log | LRV | Percent | (reported |
| Sample | Unit | Results | Re- | (reported | Pene- | by |
| Number | No | (CFUs) | sults | by Ethox): | tration | Nelson) |
| A | 1 | 4200 | 3.62 | 2.04 | 0.9130% | 2.0 |
| B | 2 | 3200 | 3.51 | 2.16 | 0.6957% | 2.1 |
| C | 3 | 2390 | 3.38 | 2.28 | 0.5196% | 2.1 |
| D | 4 | 3600 | 3.56 | 2.11 | 0.7826% | 2.1 |
| E | 5 | 3300 | 3.52 | 2.14 | 0.7174% | |
| Challenge | 6 | 4.6E+05 | 5.66 | | | |
| Control | | | | | | |
| Negative | 6 | 0 | | | | |
| Control | | | | | | |

The average Bacterial Filtration Efficiency % (BFE) for the samples was found to be 96.3%, which is an effective BFE for use in medical packaging.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1. A method for forming a nonwoven web, the method comprising:
forming a suspension of fibers into a fibrous web, the suspension comprising a cellulosic fibrous material, wherein the fibrous web has a first thickness;
forming a security image in the fibrous web, wherein the security image is defined by portions of the web having a second thickness that differs from the first thickness such that the security image can be seen by a person; and
impregnating the fibrous web with a binder composition;
wherein the nonwoven web has a Gurley porosity of from about 0.1 to about 120 seconds per 100 cubic centimeters.

2. A method as in claim 1 further comprising treating the fibrous web with a wet-strength agent.

3. A method as in claim 1, wherein the binder composition includes a carboxylated polyacrylate.

4. A method as in claim 1, wherein the binder composition further includes an opacity modifier.

5. A method as in claim 1, wherein the nonwoven web has a Gurley porosity of from about 10 to about 80 seconds per 100 cubic centimeters.

6. A method as in claim 1, wherein the nonwoven web has a Gurley porosity of from about 20 to about 60 seconds per 100 cubic centimeters.

7. A method as in claim 1, wherein the second thickness is greater than the first thickness such that the security image is a shadow mark that appears darker than the remainder of the web.

8. A method as in claim 1, wherein the first thickness is greater than the second thickness such that the security image is a watermark that appears lighter than the remainder of the web.

9. A method as in claim 1 further comprising applying an organic dye to at least one surface of the web.

10. A medical packaging substrate comprising a fibrous web formed from a cellulosic fibrous material, wherein the fibrous web has a first thickness, and wherein the fibrous web defines a security image having a second thickness that differs from the first thickness, and further wherein the fibrous web is saturated with a latex saturant, the saturated fibrous web having a Gurley porosity of from about 0.1 to about 120 seconds per 100 cubic centimeters.

11. A medical packaging substrate as in claim 10, wherein the cellulosic fibrous material is further treated with a wet-strength agent.

12. A medical packaging substrate as in claim 10, wherein the binder composition includes a carboxylated polyacrylate.

13. A medical packaging substrate as in claim 12, wherein the binder composition further includes a lower alkene polymer.

14. A medical packaging substrate as in claim 10, wherein the nonwoven web has a Gurley porosity of from about 10 to about 80 seconds per 100 cubic centimeters.

15. A medical packaging substrate as in claim 10, wherein the nonwoven web has a Gurley porosity of from about 20 to about 60 seconds per 100 cubic centimeters.

16. A medical packaging substrate as in claim 10, wherein the second thickness is greater than the first thickness such that the security image is a shadow mark that appears darker than the remainder of the web.

17. A medical packaging substrate as in claim 10, wherein the first thickness is greater than the second thickness such that the security image is a watermark that appears lighter than the remainder of the web.

18. A method for sterilizing an item, the method comprising:
sealing the item within a medical packaging substrate, wherein the medical packaging substrate is formed from a cellulosic fibrous material, wherein the cellulosic fibrous material defines a fibrous web having a first thickness, and wherein the fibrous web defines a security image having a second thickness that differs from the first thickness, and further wherein the fibrous web is saturated with a latex saturant; and
contacting the medical packaging substrate with a sterilizing gas.

19. A method for forming a medical packaging substrate, the method comprising:
forming a suspension of fibers into a fibrous web, the suspension comprising a cellulosic fibrous material, wherein the fibrous web has a first thickness;
forming a security image in the fibrous web, wherein the security image is defined by portions of the web having a second thickness that differs from the first thickness; and
impregnating the fibrous web with a binder composition; and
wherein the nonwoven web has a Gurley porosity of from about 0.1 to about 120 seconds per 100 cubic centimeters.

20. A method as in claim 19, wherein said binder composition comprises an opacity modifier.
